(19) United States
(12) Patent Application Publication
(10) Pub. No.: US 2004/0175446 A1
(43) Pub. Date: Sep. 9, 2004

(54) Modified and efficient process for the production of standardized pudina arka

(75) Inventors: Shri Gopal Agarwal, Jammu (IN); Rajinder Kumar Thappa, Jammu (IN); Bal Krishan Kapahi, Jammu (IN); Aruna Gupta, Jammu (IN); Om Parkash Suri, Jammu (IN); Ghulam Nabi Qazi, Jammu (IN)

Correspondence Address: FOLEY AND LARDNER, SUITE 500, 3000 K STREET NW, WASHINGTON, DC, 20007, US

(73) Assignee: COUNCIL OF SCIENTIFIC AND INDUSTRIAL RESEARCH

(21) Appl. No.: 10/383,257

(22) Filed: Mar. 7, 2003

(51) Int. Cl. A61K 35/78

(52) U.S. Cl. 424/747

(57) ABSTRACT

A modified and efficient single-step process for the production of standardized pudina arka as medicated water from plant Mentha herbs with ratio of drug component in arka to total arka ranging between 1:2 to 1:20 involving complete recovery of all the active material, said method comprising steps of taking aerial parts of the plant Mentha herb, crushing it optionally, adding solvent to the plant parts in the ratio ranging between 6:1 to 25:1 to obtain mixture, macerating the mixture at an elevated temperature, while maintaining desired pH, and pressure conditions, distillating the macerated mixture continuously till requisite amount of distillate is obtained, and obtaining distillate as standard pudina arka.

### FIELD OF THE PRESENT INVENTION

A modified and efficient single-step process for the production of standardized pudina arka as medicated water from plant Mentha herbs with ratio of drug component in arka to total arka ranging between 1:2 to 1:20 involving complete recovery of all the active material, said method comprising steps of taking aerial parts of the plant Mentha herb, crushing it optionally, adding solvent to the plant parts in the ratio ranging between 6:1 to 25:1 to obtain mixture, macerating the mixture at an elevated temperature, while maintaining desired pH, and pressure conditions, distillating the macerated mixture continuously till requisite amount of distillate is obtained, and obtaining distillate as standard pudina arka.

### BACKGROUND AND PRIOR ART REFERENCES

Arka or hydrosol is a liquid preparation obtained by distillation of drugs (essential molecular components having medicinal properties) soaked in water using the Arkayantra or any other convenient modern distillation apparatus [Ayurveda formulary of India, Part-I (1978) p. 21, Govt. of India, Ministry of Family Planning. Department of Health. New Delhi]. Arka is a suspension of the therapeutically important distillate in water having slight turbidity and color according to the nature of the drugs used and odor of the predominant drug.

The Indian system of medicine describes preparation and uses of various Arkas for the treatment of different ailments (Ayurvedic Materia Medica; Raghunathan. 1976; Rao. 1987: The Ayurvedic Encyclopedia. 1998). Arkas are equivalent to Aromatic waters/Medicated waters of Western medicines. Some scented waters like those of dill, aniseed, cumin, peppermint and rose etc. are being used in Ayurvedic and Unani medicines.

As per traditional methods, Arka Pudina is obtained by hydro-distillation or aqueous extraction of air dried/fresh herb (aerial parts ex. Menlha arvensis. ex. Menlha piperiia, ex. Menlha spicata and ex. Menlha viridis). It has been observed that some industrial houses also use Pudina oil (ex Menlha arvensis, ex. Menlha piperiia. ex. Menlha spicuta) and its chemical constituents for the preparation of Pudina Arka. In this procedure Cornmint oil/Peppermint oil or Spearmint oil and their chemical constituents are simply diluted with distilled water and some preservative is added. No process has been reported where in the Arka prepared has the specific physico-chemical constituents with little batch to batch variations.

The Pudina and Arka prepared from commint/Peppermint or Spearmint are the traditional Indian medicine and also being used as a food adjuvant. Literature survey has revealed that no proper scientific data is available for the standard preparation and chenwcal nature of the Arka. Under Indian System of Medicine (ISM), various Pudina preparations are being used for gastrointestinal disorders, indigestion, as stimulant and to carminative and for rheumatism and eye diseases.

Mentha arvensis. Menihti piperila, Mentha spicala and Menlha viridis oils prepared by different methods has been chemically investigated thoroughly [Guenther, E. (1948). The Essential Oils vol. III p. 587-783, D. Van Nostrand Co, Inc., NY.; Husain A. (1994) Essential Oil Plants and their Cultivation p. 103-135; Published by C1MAP. Lucknow-226015 (India): Wealth of India (Raw Materials) (1962), Vol. VI, p. 338-346. Publication & Information Directorate (CSIR). New Delhi.} but references on qualitative/quantitative chemical composition of Pudina Arka are not available.

During the preparation of different Arkas by traditional methods, various ambiguities were observed and it was found difficult to prepare Arkas of reproducible and standard quality. This may be due to the unscientific procedure adopted by the traditional practitioners/pharmacies.

As per Traditional Method for the preparation of Arka (Ayurvedic Formulary of India), coarsely grounded drug (1 part) soaked overnight in water (6 part) is distilled by using the Arkayantra or any convenient modern distillation apparatus for the recovery of Arka. According to this procedure the 1^{st} distillate and the last distillate may be discarded as these may not contain therapeutically essential principles. Pharmacopoeial standards of the preparation produced in this manner are also not known. This traditional method does not account for the amount of water added and the amount of Arka recovered.

The traditional method as reported in the Pharmacopoeia mentions the use of a fixed quantity of water for any type of drug being subjected for distillation. But the amount of water can not be the same for every drug, as the quantity of water is dependent upon the nature/bulk density and chemical composition of the drug being subjected to distillation and is a variable factor for Arka preparation. An insufficient amount of water leads to artifact formation and charring of the drug.

Due to such ambiguities and practical variations, probably Pharmacopoeia! standards of these Arkas have not been laid down. It seems that because of such difficulties, some pharmacies are using herbal oils and their chemical constituents for the preparation of Arkas.

However, some of the Ayurvedic Formulary and Pharmacopoelas of India, describe the physical characteristic like Color, Fragrance, Opacity/Transparency. Taste and Specific Gravity only of some Arkas. These texts describes Arkas as generally hazy liquids, devoid of any visible particles or fungus growth [Pharmacopoeial Standards for Ayurvedic Formulations (1976 K. Ragunathan. One of the modem reference mentions about the need of a GLC profile and have included some additional tests, if they are marketed specially for neo-natal and infants below 2 years (like the Gripewater). Such preparation may contain sugar, but prohibits the use of artificial sugar (saccharin, aspartane etc.) or any sleep inducing agents like bromides, chloral hydrate, chloroform or even alcohol (As per I.P. vol. II).

Most of the information regarding the preparation and chemical analysis on Pudina is of academic interest. No concrete information for the quantitative preparation/isolation and Standardization of Arka is available in the literature, hence the necessity for development of such a standardized process.

Present study was undertaken to develop methods of production and Pharmacopoeial standards for Arkas used in the Indian System of Medicine (Ayurvedic. Unam and Siddha Drugs) and Preparation of Monographs for The Ayurvedic pharmacopoeia, as no suitable methods are available in literature.

### OBJECTS OF THE PRESENT INVENTION

The main object of the present invention is to develop a process for the production of standardized pudina arka.

Another main object of the present invention is to develop one-step process for the production of standardized pudina arka.

Yet another object of the present invention is to develop a standardized pudina arka with ratio of drug component in arka to total arka ranging between 1:2 to 1:20.

Still another object of the present invention is to develop a standardized pudina arka by one-step process involving complete recovery of all the active material.

Still another object of the present invention is to develop a process of obtaining standardized pudina arka with solvent to the plant parts in the ratio ranging between 6:1 to 25:1.

Still another object of the present invention is to develop standardized arka capable of being stored at temperature ranging between 60 to −10° C.

Still another object of the present invention relates to process of producing pudina arka without formation of any side-products.

### SUMMARY OF THE PRESENT INVENTION

A modified and efficient single-step process for the production of standardized pudina arka as medicated water from plant Mentha herbs with ratio of drug component in arka to total arka ranging between 1:2 to 1:20 involving complete recovery of all the active material, said method comprising steps of taking aerial parts of the plant Mentha herb, crushing it optionally, adding solvent to the plant parts in the ratio ranging between 6:1 to 25:1 to obtain mixture, macerating the mixture at an elevated temperature, while maintaining desired pH, and pressure conditions, distillating the macerated mixture continuously till requisite amount of distillate is obtained, and obtaining distillate as standard pudina arka.

### DETAILED DESCRIPTION OF THE PRESENT INVENTION

Accordingly, the present invention relates to a modified and efficient single-step process for the production of standardized pudina arka as medicated water from plant Mentha herbs with ratio of drug component in arka to total arka ranging between 1:2 to 1:20 involving complete recovery of all the active material, said method comprising steps of:

taking aerial parts of the plant Mentha herb,

crushing it optionally,

adding solvent to the plant parts in the ratio ranging between 6:1 to 25:1 to obtain mixture,

macerating the mixture at an elevated temperature, while maintaining desired pH, and pressure conditions,

distillating the macerated mixture continuously till requisite amount of distillate is obtained, and

obtaining distillate as standard pudina arka.

In still another embodiment of the present invention, wherein Mentha is selected from a group comprising Mentha arvensis, Mentha piperita, Mentha spicata, and Mentha viridis.

In still another embodiment of the present invention, wherein Mentha herb can be air dried or fresh.

In still another embodiment of the present invention, wherein the mixture is agitated throughout the process.

In still another embodiment of the present invention, wherein solvent can be selected from a group comprising water, steam, alcohol, and superheated steam.

In still another embodiment of the present invention, wherein temperature is ranging between 60° C. to 160° C.

In still another embodiment of the present invention, wherein pressure is ranging between 5 atmosphere to 200 atmosphere.

In still another embodiment of the present invention, wherein pH is ranging between 4 to 8.

In still another embodiment of the present invention, wherein the said process can be carried out in an atmosphere selected from a group comprising natural, inert, nitrogen, and vacuum atmosphere.

In still another embodiment of the present invention, wherein the time duration for the maceration ranging between 30 minutes to 24 hours.

In still another embodiment of the present invention, wherein the arka obtained is stored at temperature ranging between 60 to −10° C.

In still another embodiment of the present invention, wherein said process leads to formation of arka with consistent physio-chemical parameters and standard active molecular composition.

In still another embodiment of the present invention, wherein the arka obtained from the said process is of pharmaceutical, and flavoring grade.

In still another embodiment of the present invention, wherein said process has helped overcome the problem extraction of arka due to essential oils of the drugs that are insoluble in water.

In still another embodiment of the present invention, wherein said process does not lead to formation of any side-products.

In still another embodiment of the present invention, wherein said process leads to formation of arka that is free of burnt and off odor.

In still another embodiment of the present invention, wherein optimal levels of temperature, pressure, solvent, time, and activity are interrelated to produce desired results.

In still another embodiment of the present invention, wherein said process escapes the formation of undesirable chemicals, fibrous, decomposed, artifacts, an other side products.

In still another embodiment of the present invention, wherein the arka shows essential components of plant Mentha arvensis comprising 1 -limonene in the concentration ranging between trace and 0.5%, menthone ranging between 6-8%, isomenthone ranging between 2.5-4.5%, and menthyl acetate ranging between 1.6-2.5%, and 1-menthol ranging between 80-84%.

In still another embodiment of the present invention, wherein the arka shows essential components of plant Mentha piperita comprising 1-limonene in the concentration ranging between 0.5-2.5%, menthone ranging between 6-12%, menthyl acetate ranging between 1.0-3.5%, and 1-menthol ranging between 50-60%.

In still another embodiment of the present invention, wherein the arka shows essential components of plant Mentha spicata comprising 1-limonene in the concentration ranging between 8-12%, and 1-carvone ranging between 50-67%.

In still another embodiment of the present invention, wherein the arka shows essential components of plant Mentha viridis comprising piperitenone oxide of concentration ranging between 78-86%.

The present invention relates to a modern and improved process for the production of Pudina Arkas (Pudina Arka ex. Mentha arvensis, Pudina Arka ex. Mentha piperita, Pudina Arka ex. Mentha spicata and Pudina Arka ex. Mentha viridis). The invention particularly relates to a novel process and standardized product for the production of Pudina Arka of different varieties based on starting herbal Pudina. More particularly this invention relates to the direct extraction or” air dried/fresh Pudina herb (aerial parts) in a single step into arka of high and standard quality, in varied solvent media under optimum conditions of temperature, pressure, pH and time. Standard volume of Arka is separated from the undesirable, fibrous matter for the recovery of flavoring/pharmaceutical grade Pudina Arka of natural flavor with specific Physicochemical constants and active molecular constituents characteristic of each variety.

We have developed an efficient extraction method for the preparation of Arka. We have also developed methods for the quantitative estimation of active organic molecules of Arka. as well TLC and GLC method for identification and quantification of important chemical constituents for the standardization of Pudina Arka. The present patent reports some new and important findings which emerged out of this work along with proposed Pharmacopoeial standards.

Protocol for the preparation of Arka was designed keeping in view the various ambiguities, encountered in traditional methods. Thus Modern Method (Developed by us at RRL. Jammu) accounts for specific drug to solvent ratio, specific drug to Arka ratio. Grinding and soaking parameters. 1^{st} distillate and Last distillate problems, artifacts formation. Quality parameters and Pharmacopoeial standards.

Protocol for the Standardization of Arka was designed keeping the general ISO requirements as a minimum base which included 1. Definition; 2. Procedure for the Preparation of Arka: 3. Assay: 4. Assay Procedure; 5. Literature; 6. Characteristic Constituent Identification; 7. Characteristics of Arka [Description which includes Color. Fragrance. Opacity/Transparency. Taste and Any other peculiarity]; 8. Identification [which includes Content of essential oil in Arka: Minimum/Maximum. Specific Gravity of Arka (Not characteristic of preparation). Specific Gravity of essential oil obtained from Arka (Characteristic of preparation). Optical Rotation of Arka (Not characteristic of preparation), Optical Rotation of essential oil obtained from Arka (Characteristic of preparation), Refractive Index of essential oil obtained from Arka (Characteristic of preparation), Content of characteristic major Constituents]; 9. TLC Analysis of Oil obtained from Arka with photographs [type of TLC plates. Test solution. Reference solution, quantity of test and reference samples and development path length. Solvent System and Detection/visualization reagents. Rf Values and color (Major spots)]: 10. Gas Chromatography Analysis of Oil from Arka with GC graphs [Test sample. Gas Chromatograph Model. Column & Stationary phase. Carrier Gas, Column Temperature. Injection port Temperature. Detector Temperature, Recorder. & Chart speed, Sample size. Elution pattern. Retention time. % of Constituents, The identification of compounds was done by comparing the retention time of peaks and by peak enrichment technique with standard samples run under similar operating conditions. The retention time of the principal peaks in the chromatogram obtained with the test solution is similar to that of the principal peaks in the chromatogram obtained with the reference solution. Overall chromatogram is in comparison with standard chromatographic profile]; 11. Sampling [Minimum mass of laboratory sample required] and 12. References. (Note: Minimum 1 ml of oil is required for determination of Specific Gravity and Optical Rotation. Specific Gravity determined with Pyknometer of capacity in the range of 0.2 ml and 0.5 ml; Optical Rotation determined neat or as about 10% methanolic solution).

The plant Mentha arvensis Fam. Lamiaceae). an annual herb with rhizomes is a native of Japan and now cultivated in various parts of the world from South America. Europe to China. Today India is the major supplier to the world market (approx. 20.000 tones of oil per annum). Menlha piperita, Mentha spicata and Mentha viridis are also being cultivated on large scale either for the production of oils or to be used as culinary herbs. Mentha aquatica L. (Marsh Mint). Mentha longifolia L. (Jungli Mint), Mentha pulegium L. (Pennyroyal Mint) and Mentha rotundifolia are also being used at some places for the same purposes, although were found to have different chemical composition. Successful and significant penetration of the international market will be dependent upon introduction of quality products by India in the form of value added newer products. This patent reports a new process for the standardized Pudina Arka to be used by ‘over the counter natural food additives’ and pharmaceutical industry.

One of the problems faced by the Arka industry is that the most of drugs (essential molecular components having medicinal properties) of the it uses, contain essential oils which are insoluble in water and this makes their extraction difficult. The present invention provides a process which uses food grade solvents with total recovery of desirable materials in quantitative yield without solvent residues along with the separation of value added fraction of high utility from starting materials.

Therefore, invention of a direct and economical extraction/distillation process for the recovery of total Arka with complete active materials without decomposition during processing in a single step without side products is advantageous, urther. the starting material being a natural Agricultural commodity, of Indian origin at required place, the final product is of high flavor and pharmaceutical value and is free from decomposed impurities and off odors.

The main objective of the present invention is to provide an improved process for the production of value added Pudina-Arka from Pudina herb which obviates the drawbacks of the present methods as detailed above.

Another objective of the invention is to use a novel solvent i.e. water and steam in such a way which will only extract the desirable materials without affecting the decomposition/degradation or charring of the desired molecules so as to give the maximum yield of the product.

Yet still another objective is that by employing these solvents and the conditions mentioned in the invention, the actual drug to water ratio and drug to Arka ratio and chemical composition is standardized for each variety

Yet another objective of the invention is to account for the standard recovery of active constituents of the drug during preparation of Arkas for each variety. The composition of distillate was established at different stages of extraction-distillation process from different batches of drug, procured from different regions and drug markets. Based on these elaborate chemical studies the drug to water and drug to Arka ratio and recovery time etc. were fixed to obtain Arka of uniform chemical composition specific of Pudina variety

Arka is a suspension of the therapeutically important distilled essential oil in water having slight turbidity with cloudy-whitish tinge and pleasingly fresh—mint reminiscent of cornmint or peppermint or spearmint or of other variety. Due to turbid and hazy nature of the Arka, the composition of suspended oil and water soluble oil were established separately and based on such critical observations various parameters for the production of quality material were conclusively established for each variety.

Further, these studied helped in establishment of reproducible Pharmacopoeial standards for these drugs

Accordingly the present invention provides an improved process for the production of standardized Pudina Arka (medicated water) which comprises direct extraction and distillation of air dried or fresh Pudina Herb of different varieties (aerial parts) individually as such or in crushed/grounded form, in a single step with food grade solvents such as water, steam or superheated steam, with or without prior water soaking, at elevated temperature, under natural/inert/vacuum atmosphere, at a desired pH and pressure, with characteristic and specific drug to water ratio and specific drug to Arka ratio, recovering and separating the specific volume of product without any charring or artifacts formation with consistent physico-chemical parameters and standard active molecular composition characteristic for each variety.

In an embodiment of the present invention the solvents used may be such as water, steam, superheated steam and even alcohol, from any source. Water soaking of drug 24 hours earlier is not essential but soaking does not make any difference. Further, contrary to Traditional Method for the preparation of Arkas, 1^{st} distillate should not be discarded as it consists of essential principles in large excess and the distillation is monitored for complete recovery of therapeutically essential principles. Quality parameters maintained during preparation did not allow, formation of artifacts. Such a practical extraction/distillation method provided arka of consistent quality which helped in formulating Pharmacopoeial standards for the Pudina Arka ex. Mendta arvensis. Pudina Arka ex. Mentha piperita Pudina Arka ex. Mentha spicata and Pudina Arka ex. Mentha viridis etc.

The process for the production of Pudina Arka with complete recovery of active ingredients comprises of the direct extraction/distillation of Pudina herb in a single step with food grade solvents under continuous maceration and agitation without any decomposition of natural constituents, in varied solvent media if desired, under optimum conditions of temperature in the range of 160 to 60° C., under natural/inert/Nitrogen/Vacuum atmosphere at a pH in the range of 4 to 8, for a period in’ the range of 30 min. to 24 hr, drug to water ratio, 1:6 to 1:25. drug to arka ratio 1:2 to 1:20, recovering and separating the product and storing at a temperature in the range of 60 to −10° C. in inert/vacuum/pasteurized atmosphere with or without preservatives.

In general the usual relationship of drug to water ratio, drug to arka ratio, monitoring of 1^{st} & Last distillate for complete recovery of therapeutically essential principles. Quality parameters during preparation, time, temperature, and amount apply in the present case. Preferred temperatures range of 160 to 60° C. has been found to be ideal. At lower temperature extraction tends to be slow unless a high ratio of raw material to solvent is utilized.

In general the usual relationship of activity, temperature, pressure, medium and time, along with other physical and chemical parameters apply in the present case. It is important to optimize all the above mentioned conditions in order to get the desired product of uniform standard quality.

The invention is described further with reference to examples given below. The example should not be construed as to restrict the scope of the invention

### EXAMPLE 1

Japanese Pudina herb coarsely crushed (100 g.) and water (2000 ml i.e. drug to water ratio 1:20) were mixed and macerated at 60° C. efficient) for 4 hrs. and placed in a round bottom flask of 3.0 lit capacity. The mixture was allowed to boil at atmospheric pressure while maintaining the pH at 6.5 to 7.5 and distillate (1500 ml.) condensed by circulating water and recovered (i.e. drug to arka ratio 1:15).

### EXAMPLE 2

Crushed Mentha spicata herb (1.0 kg.) along with water (6000 ml-) was placed in a round bottom flask of 20.0 lit capacity. The hot steam (8.0 lit; i.e. drug 10 water ratio 1:14) of pH 7.0 was allowed to pass and distillate (7.0 lit) alter proper condensation was recovered (i.e. drug to arka ratio 1:7).

### EXAMPLE 3

Air dried Peppermint herb (300 g.; ex. Mentha piperita) and distilled water (4800 ml.; i.e. drug to water ratio 1:16) were mixed and macerated efficiently and placed in a Round bottom flask of 10.0 lit capacity. The mixture was allowed to boil at 720 mm and distillate (3.0 I.I recovered by utilizing any convenient modern distillation assembly (i.e. drug to arka ratio 1:10).

### EXAMPLE 4

Finally grounded Meetha Pudina herb (500 g. ex. Mentha virdis) was soaked in distilled water (4000 ml) for 24 hours and macerated. Afterwards Modified Clevenger apparatus connected to the Round bottom flask with addition of 5000 ml. of fresh water. The mixture was allowed to boil at 2.0 atmospheric pressure and distillate (6.0 I.) recovered by utilizing any convenient modern distillation assembly (i.e. drug to arka ratio 1:12).

Proposed Pharmacopoeial standards for the Standardization of Japanese Pudina Arkas:

Based on above discovery, the following Pharmacopoeial standards for the Standardization of Japanese Pudina Arka was designed keeping the general ISO requirements as a minimum base.

1. Definition:

Japanese Pudina Arka (or Cornmint Arka) is a liquid preparation obtained by hydro-distillation of air dried Japanese Pudina leaves along with overground parts of the plant (Mentha arvensis) in water by using any convenient modern distillation apparatus.

2. Assay:

Japanese Pudina Arka (Mentha arvensis) contain not less than 0.14% of essential oil (Arka with floating oil droplets). However, its percentage usually varies from 0.14% to 0.18% in samples prepared from different varieties of Japanese Pudina (Cornmint leaves) procured from different sources. Dissolved essential oil in Japanese Pudina Arka without floating oil droplets happens to be 0.10%. Pudina collected from different regions on hydro-distillation yielded 2.8 to 3.4 percent of essential oil.

3. Assay Procedure

Hydro-distillation: Place 500 ml of well stirred Japanese Pudina Arka in a round bottom, standard joint flask of 1.0 liter capacity. Attach the proper Clevenger type apparatus and the condenser to the flask and add enough water to fill the trap. Place the flask in heating mantle to heat to approximately 130° C. Adjust the temperature control and continue the distillation for 3 hrs. until no further increase in oil is observed. Allow the oil to stand at completion, for good separation of oil and determine the number of c.c. of oil obtained and express the yield as a v/v percentage i.e. number of c.c. of oil per 100 ml of Japanese Pudina Arka. Separate the oil from water and dry over anhydrous sodium sulphate.

4. Literature:

Chemical constituents of Arka has not been reported in literature. However, the essential oil obtained by distillation of cornmint leaves is reported to contain limonene, 1-menthol (60-90%), menthone (7-20%), menthyl acetate and others.

5. Characteristic Constituent Identified

Essential oil obtained from Arka is a mixture consisting of at least one characteristic major compound and more than twelve minor compounds. Major characteristic compound was identified as 1-menthol (80-84% of oil), menthone (6-8%). menthyl acetate 0.6-2.5%) vide TLC GLC and spectroscopic techniques (details and graph enclosed).

6. Characteristics

Arka is a suspension of the therapeutically important distilled essential oil in water having slight turbidity.

6.1.1 Color:turbid, cloudy-whitish tinge

6.1.2 Fragrance:pleasingly fresh—minty reminiscent of cornmint.

6.1.3 Opacity/Transparency:cloudy with slight turbidity and white tinge (on standing turns colorless with separation of oil at the top)

6.1.4 Taste:minty, slightly bitter with cooling sensation reminiscent of cornmint.

6.1.5 Any other peculiarity:watery touch with drops of oil floating on the surface

7. Identification

7.1 Content of essential oil in Arka

| | | | |
|---|---|---|---|
| Minimum: | 0.14% v/v | Maximum | 0.18% v/v |

7.2 Specific Gravity of Arka at 32° C.:Range 0.9831 to 1.00

7.3 Specific Gravity of essential oil obtained from Arka at 32° C.:Range 0.8818 to 0.9008

7.4 Optical Rotation of essential oil obtained from Arka at 32° C.: Range −38.8° to −41.5°

7.5 Refractive Index of essential oil obtained from Arka at 32° C. 1.44 to 1.45

7.6 Content of characteristic major Constituents

1-menthol:80-84%

menthone:6-8%

isomenthone:2.5-4.5%

menthyl acetate:1.6-2.5%

(Note: Minimum 1 ml of oil is required for determination of Specific Gravity and Optical Rotation. Specific Gravity determined with Pyknometer of capacity in the range of 0.2 ml and 0.5 ml; Optical Rotation determined as about 2-10% methanolic solution).

8. TLC Analysis of Oil Obtained from Japanese Pudina Arka (Mentha Arvensis)

8.1 TLC analysis was carried out on aluminium plates precoated with Silica gel 60.

8.2 Test solution:Dissolve 0.1 ml of the oil to be examined in 1 ml. of toluene or in any other suitable solvent.

8.3 Reference solution:Dissolve 0.1 g. of the 1-menthol in 1 ml. of” toluene or in any other suitable solvent.

8.4 Apply separately to the plate as bands 2 \hat{ } 1 of the reference solution and 4 μl of the test solution and develop over a path of 15 cm.

8.5 Solvent System:Methanol:Benzene (5:95)

8.6 Detection:

i. Spray with 2% Vanillin-Sulfuric Acid solution and examine in day light for 5 to 10 min while heating at 100 to 105° C.;

ii. Spray with Anisaldehyde-Sulfuric Acid solution and examine in day light for 5 to 10 min. while heating at 100 to 105° C.; 8.7 Rf Values (Major spots):

0.15 1-menthol (i. dark Maroonish-purple colored spot:ii. dark bluish-purple),

0.30 menthone (i. dark bluish-purple colored spot; ii. pinkish-purple),

0.45 menthyl acetate (i. dark Maroonish-purple colored spot ; ii. dark bluish-purple).

9. Gas Chromatography Analysts of Oil from Japanese Pudina Arka

Test sample:Essential oils obtained from Japanese Pudina Arka (Mentha arvensis)

Gas Chromatograph Model NUCON-5765

Column & Stationary phase:30 m fused silica capillary column walls coated with FFAP

Carrier Gas:Helium, 1.5 ml. min’¹

Column Temperature:90° C. for 2 min. then programmed at the rate of 7”C min’¹ to 220° C.

Injection port Temperature:220° C.

Detector Temperature:260° C.

Recorder:2 mV, signal attenuation 1:100

Chart speed:1 cm,min’

Sample size:0.10 pl (For GC analyses, pure (0.1 pl) is injected with a 1.0 yl syringe).

| Elution pattern: | Retention time | % of Constituents |
|---|---|---|
| 1-limonene | (Rt. 6.37 min.) | Trace-0.5% |
| menthone | (Rt. 10.81 min.) | 6.0-8.0% |
| isomenthone | (Rt. 11.30 min.) | 2.5-4.5% |
| metnhyl acetate | (Rt. 12.82 min.) | 1.6-2.5% |
| 1-menthol | (Rt. 13.97 min.) | 80.0-84.0% |

The identification of compounds was done by comparing the retention time of peaks and by peak enrichment technique with standard samples run under similar operating conditions. The retention time ot” the principal peaks in the chromatogram obtained with the test solution is similar to that of the principal peaks in the chromatogram obtained with the reference solution. Overall chromatogram is in comparison with standard chromatographic profile.

10. Sampling:

Minimum mass of laboratory sample: In order to carry out all the test methods described in this Standard, a sample of at least 1.0 liter Arka is required.

However, if Specific Gravity and Optical Rotation are omitted in special cases (where less sample is available), 100 ml sample may be sufficient to carry out all other tests.

Proposed Pharmacopoeial standards for the Standardization of Spearmint Arkas:

Spearmint Pudina Arka is a liquid preparation obtained by hydro-distillation of air dried Pudina leaves along with overground parts of the plant (Mentha spicata) in water by using any convenient modern distillation apparatus.

2. Assay:

Pudina Arka (ex. Mentha spicata) contain not less than 0.15% of essential oil (Pudina Arka with floating oil droplets). However, its percentage usually vanes from 0.15% to 0.18% in samples prepared from different varieties of Pudina (spearmint leaves procured from different sources. Dissolved essential oil in Pudina Arka without floating oil droplets happens to be 0.10-0.13%. Pudina collected from different regions on hydro-distillation yielded 1.0 to 1.54 percent of essential oil.

3. Literature:

Chemical constituents of Arka has not been reported in literature. However, the essential oil obtained by distillation of spearmint leaves is reported to contain 1-limonene, linalool, 1,8-cineole, 1-carvone (major) and others.

4. Characteristic Constituent Identified

Essential oil obtained from Arka is a mixture consisting of at least one characteristic major compound and more than ten minor compounds. Major characteristic compound was identified as 1-carvone (65-78% of oil) vide TLC, GLC and spectroscopic techniques (details and graph enclosed).

5. Characteristics:

Arka is a suspension of the therapeutically important distilled essential oil in water having slight turbidity.

5.1 Description

5.1.1 Color:cloudy-whitish tinge

5.1.2 Fragrance:pleasingly sweet-fruity fresh and spicy-minty reminiscent of spearmint.

5.1.3 Opacity/Transparency:cloudy with slight turbidity and white tinge

5.1.4 Taste:sweetish-fruity-spicy, slightly bitter reminiscent of spearmint.

5.1.5 Any other peculiarity: watery touch with drops of oil floating on the surface

6 Identification:

6.1 Content of essential oil in Arka:

| | | | |
|---|---|---|---|
| Minimum: | 0.15% v/v | Maximum: | 0.18% v/v |

6.2 Specific Gravity of Arka at 22° C.: Range 0.9957-0.100

6.3 Specific Gravity of essential oil obtained from Arka at 22° C.: Range 0.93-0.9450

6.4 Optical Rotation of essential oil obtained from Arka at 22° C.: Range −45.20 to 48.750

6.5 Refractive Index of essential oil obtained from Arka at 20° C.: 1.48 to 1.500

6.6 Content of characteristic major Constituents

| | |
|---|---|
| 1-limonene | 8.0-12.0% |
| 1-carvone | 50.0-67.0% |

(Note: Minimum 1 ml of oil is required for determination of Specific Gravity and Optical Rotation. Specific Gravity determined with Pyknometer of capacity in the range of 0.2 ml and 0.5 ml; Optical Rotation determined as about 10% methanolic solution).

7. TLC Analysis of Oil Obtained from Pudina Arka (ex. Mentha spicatd)

7.1 TLC analysis was carried out on aluminum plates precoated with Silica gel 60 and Silica gel 60 F254 (0.2 mm, Merck)

7.2 Test solution:Dissolve 0.1 ml of the oil to be examined in 1 ml. of toluene or in any suitable solvent.

7.3 Reference solution:Dissolve 0.1 g. of the 1-carvone in 1 ml. of toluene or in any suitable solvent.

7.4 Apply separately to the plate as bands 2 μl of the reference solution and 4 μl of the test solution and develop over a path of 15 cm.

7.5 Solvent System:Ethyl acetate: Hexane (25:75)

7.6 Detection:

i. Spray with 2% Vanillin-Sulfuric Acid solution and examine in day light for 5 to 10 min while heating at 100 to 105° C.;

ii. Spray with Anisaldehyde-Sulfuric Acid solution and examine in day light for 5 to 10 min while heating at 100 to 105° C.;

ii. Keep in a chamber filled with Iodine vapors for 5 to 10 min and examine in day light; and

iv. Spray with 2:4-Dinitrophenylhydrazine solution.

7.7 Rf Values (Major spots). 0.69 (i. bluish-purple colored spot; ii. Pinkish-purple). 0.62 (i. dark blue colored spot; ii. bluish-purple), 0.52 Carvone (iv. Yellowish-orange colored spot; i. Purple with yellow background; ii Reddish-orange with yellow tinge; iii. Dark yellow spot), 0.43 (i. dark blue; ii. bluish-purple), 0.34 (i. dark blue; ii. bluish-purple).

8. Gas Chromatography Analysis of Oil from Pudina Arka (ex. Mentha spicata)

Test sample:Essential oils obtained from Pudina Arka (ex. Mentha spicata)

Gas Chromatograph Model NUCON-5765

Column & Stationary phase:30 m fused silica capillary column walls coated with FFAP

Carrier Gas:Helium, 1.5 ml. min’¹

Column Temperature:90° C. for 2 min. then programmed at the rate of 7° C. min’ to 220° C.

Injection port Temperature:’ 220° C.

Detector Temperature:’ 260° C.

Recorder:2 mV, signal attenuation 1:100

Chart speed:1 cm.min”¹ ”

Sample size:0.10 |il (For GC analyses, pure (0.1\hat{ }1) is injected with a 1.0 \hat{ }1 syringe).

| Elution pattern: | Retention time | % of Constituents |
|---|---|---|
| 1-limonene | (Rt. 7.43 min.) | 8.0-12.0% |
| 1-carvone | (Rt. 17.40 min.) | 50.0-67.0% |

The identification of compounds was done by comparing the retention time of peaks and by peak enrichment technique with standard samples run under similar operating conditions. The retention time of the principal peaks in the chromatogram obtained with the test solution is similar to that of the principal peaks in the chromatogram obtained with the reference solution. Overall chromatogram is in comparison with standard chromatographic profile.

11. Sampling

Minimum mass of laboratory sample: In order to carry out all the test methods described in this Standard, a sample of at least 1.0 lit Arka is required.

However, if Specific Gravity and Optical Rotation are omitted in special cases (where less sample is available), 100 ml sample may be sufficient to carry out all other tests.

Gas Chromatography Analysis of Oil from Pudina Arka (ex. Mentha piperita)

Experimental Conditions Same as Above:

| Elution pattern: | Retention time | % of Constituents |
|---|---|---|
| 1-limonene | (Rt. 6.37 min.) | 0.5-2.5% |
| menthone | (Rt. 10.81 min.) | 6.0-12.0% |
| menthyl acetate | (Rt. 12.82 min.) | 1.0-3.5% |
| 1-menthol | (Rt. 13.97 min.) | 50.0-84. |

Gas Chromatography Analysis of Oil from Pudina Arka (ex. Mentha viridis)

Experimental Conditions Same as Above:

| Elution pattern: | Retention time | % of Constituents |
|---|---|---|
| piperitenone oxide | (Rt. 21.12 min.) | 78.0-86.0% |

Main Advantages of the Present Invention

1. A modem reproducible one step standardized process for the production of Pudina Arka of uniform quality as per standards appended on last pages of this invention for Pudina Arka ex. Mentha arvensis, Pudina Arka ex. Mentha piperita, Pudina Arka ex. Mentha spicata and Pudina Arka ex. Mentha vmdis.

2. An improved process which provides the product of uniform quality without the presence of any decomposed materials or artifacts, in contrast to traditional process wherein the quality of arka is not reproducible and very poor due to charring and artifacts formation.

3. An improved process for the production of Pudina Arka where drug to water ratio and drug to Arka ratio have been standardized to provide Arka of uniform reproducible quality with relation to physico-chemical parameters and active molecules composition for each variety.

The invention provides an economical extraction cum distillation process for the preparation of fixed quantity of Arka per unit of plant material with complete recovery of therapeutically important active materials, which is not possible with traditional process.

1. A modified and efficient single-step process for the production of standardized pudina arka as medicated water from plant Mentha herbs with ratio of drug component in arka to total arka ranging between 1:2 to 1:20 involving complete recovery of all the active material, said method comprising steps of:
a. taking aerial parts of the plant Mentha herb,
b. crushing it optionally,
c. adding solvent to the plant parts in the ratio ranging between 6:1 to 25:1 to obtain mixture,
d. macerating the mixture at an elevated temperature, while maintaining desired pH, and pressure conditions,
e. distillating the macerated mixture continuously till requisite amount of distillate is obtained, and
f. obtaining distillate as standard pudina arka.

2. A process as claimed in claim 1, wherein Mentha is selected from a group comprising Mentha arvensis, Mentha piperita, Mentha spicata, and Mentha viridis.

3. A process as claimed in claim 1, wherein Mentha herb can be air dried or fresh.

4. A process as claimed in claim 1, wherein the mixture is agitated throughout the process.

5. A process as claimed in claim 1, wherein solvent can be selected from a group comprising water, steam, alcohol, and superheated steam.

6. A process as claimed in claim 1, wherein temperature is ranging between 60° C. to 160° C.

7. A process as claimed in claim 1, wherein pressure is ranging between 5 atmosphere to 200 atmosphere.

8. A process as claimed in claim 1, wherein pH is ranging between 4 to 8.

9. A process as claimed in claim 1, wherein the said process can be carried out in an atmosphere selected from a group comprising natural, inert, nitrogen, and vacuum atmosphere.

10. A process as claimed in claim 1, wherein the time duration for the maceration ranging between 30 minutes to 24 hours.

11. A process as claimed in claim 1, wherein the arka obtained is stored at temperature ranging between 60 to −10° C.

12. A process as claimed in claim 1, wherein said process leads to formation of arka with consistent physio-chemical parameters and standard active molecular composition.

13. A process as claimed in claim 1, wherein the arka obtained from the said process is of pharmaceutical, and flavoring grade.

14. A process as claimed in claim 1, wherein said process has helped overcome the problem extraction of arka due to essential oils of the drugs that are insoluble in water.

15. A process as claimed in claim 1, wherein said process does not lead to formation of any side-products.

16. A process as claimed in claim 1, wherein said process leads to formation of arka that is free of burnt and off odor.

17. A process as claimed in claim 1, wherein optimal levels of temperature, pressure, solvent, time, and activity are interrelated to produce desired results.

18. A process as claimed in claim 1, wherein said process escapes the formation of undesirable chemicals, fibrous, decomposed, artifacts, an other side products.

19. A process as claimed in claim 1, wherein the arka shows essential components of plant Mentha arvensis comprising 1-limonene in the concentration ranging between trace and 0.5%, menthone ranging between 6-8%, isomenthone ranging between 2.5-4.5%, and menthyl acetate ranging between 1.6-2.5%, and 1-menthol ranging between 80-84%.

20. A process as claimed in claim 1, wherein the arka shows essential components of plant Mentha piperita comprising 1-limonene in the concentration ranging between 0.5-2.5%, menthone ranging between 6-12%, menthyl acetate ranging between 1.0-3.5%, and 1-menthol ranging between 50-60%.

21. A process as claimed in claim 1, wherein the arka shows essential components of plant Mentha spicata comprising 1-limonene in the concentration ranging between 8-12%, and 1-carvone ranging between 50-67%.

22. A process as claimed in claim 1, wherein the arka shows essential components of plant Mentha viridis comprising piperitenone oxide of concentration ranging between 78-86%.
